(19) United States
(12) Patent Application Publication
(10) Pub. No.: US 2013/0184233 A1
(43) Pub. Date: Jul. 18, 2013

(54) Personal Lubricant Compositions

(71) Applicants: Catherine Ann Carter, Carrolton, TX (US); Alexander Vickers, Irvine, TX (US)

(72) Inventors: Catherine Ann Carter, Carrolton, TX (US); Alexander Vickers, Irvine, TX (US)

(21) Appl. No.: 13/740,409

(22) Filed: Jan. 14, 2013

Related U.S. Application Data
(60) Provisional application No. 61/586,378, filed on Jan. 13, 2012.

(51) Int. Cl. A61K 8/73; A61Q 19/00

(52) U.S. Cl. CPC A61K 8/737; A61Q 19/00; B65D 35/46
USPC 514/52; 514/54

(57) ABSTRACT

A skin lubrication lotion that functions as a skin rubbing anti-friction barrier compound to prevent skin rashes from burning of skin from excessive rubbing, said lotion comprising: a lubricant; a skin numbing ingredient including lidocane; a vitamin ingredient, wherein the vitamin is selected from the group consisting of vitamin A, C, E, D or B 12; an ingredient that causes human skin to tingle, wherein the tingling ingredient is selected from the group consisting of menthol and L-arginine ethyl ester; wherein the skin lotion further consisting essentially of about: 71% water, 0.070% sodium benzoate, 0.070% potassium Sorbate, 0.180% citric acidanhydrous (FCC, USP), 14.200% glycerine 99.7%, 14.200% propylene glycol USP, 0.140% Xanthan gun 80 mesh, and 0.140% guar gun 250 (food grade).

This application claims priority to U.S. Provisional Patent Application to Alexander Vickers entitled “PERSONAL LUBRICANT COMPOSITIONS AND DISPENSING MECHANISM”, Ser. No. 61/586,378, filed Jan. 13, 2012, the disclosure of which is hereby incorporated entirely herein by reference.

### BACKGROUND OF THE INVENTION

1. Field of the Invention

The present disclosure is directed to a formula for a personal topical body lotion and the method of combining components of the disclosed formula, wherein the lotion is applied using a mechanism for dispensing the disclosed lotion. The formula is dispensed using a bag on valve (“BOV”) delivery system. The BOV delivery system allows the disclosed lubricant to be sprayed in any direction from any angle.

2. Description of the Related Art

It is well known to in the art to apply creams and lotions to the human body for many reasons, such as for skin smoothing and hydration. It is also well known in the art to provide devices to enhance the application of the lotion to the human body by way of various spray devices, including bag on valve designs. The following patents are provided are provided as examples of such known art, and are herein incorporated by reference for their supporting teachings to the disclosed invention, whereby:

U.S. Pat. No. 5,179,982, by Berube et al., is an apparatus for discharging a fluid and more particularly, for spraying a liquid.

US Patent App. 2011/0006076 to Williams, is a system for dispensing sprayable material.

US Patent App. 2008/0206156 to Cronk, is a continuous spray scalp therapy and dispensing system for same.

U.S. Pat. No. 3,421,698, on Jan. 14, 1969, to W. Baltzer, is a device for spraying liquids by means of a propellant.

U.S. Pat. No. 5,208,031, to Kelly, is a sexual lubricant containing zinc as an anti-viral agent.

U.S. Pat. No. 5,341,968, to Vandonicck, is a spray can incorporating a discharge pressure regulating system.

US Patent App. 2008/0193489, to DeAnnond et al., is a personal lubricant composition that is free of glycerin and paraffin's.

US Patent App. 2009/0088405, to Kehoe et al., is a personal lubricant with built-in or accompanying odorant delivery systems.

U.S. Pat. No. 4,851,434 (the '434 patent) to Deckner discloses a non-greasy, non-irritating moisturizer, and compositions containing as the major moisturizing component the di-, tri- or polyglycol amide or glucamine reaction product with an α-hydroxy-substituted fatty acid, with the formula containing hydrogen or a lower alkyl, preservatives, thickeners, skin-soothing agents, such as allantoin and/or dl-panthenol, and water. The non-irritating moisturizer disclosed in the '434 patent does not produce a warming effect when it contacts body-generated moisture and does not spread spontaneously on latex and human skin.

U.S. Pat. No. 4,952,560 to Kigasawa et al. discloses an ointment base containing a water-soluble protein consisting of gelatin, casein and soybean protein and a monohydric alcohol with a carbon number of 2 to 4 and/or an oleginous substance and additionally containing a wetting agent selected from the group consisting of an alkylene glycol containing 2 to 6 carbon atoms, polyethylene glycol having an average molecular weight of about 200 to 800, glycerin, trimethylolpropane and sorbitol, in a range of 1 to 35 weight % based on the weight of the whole ointment. While the ointment base improves percutaneous absorption of drugs, it is too thick to function as a lubricant.

U.S. Pat. No. 5,441,723 to Simmons discloses a non-toxic hypocompatible biodegradable germicide. The non-toxic hypocompatible biodegradable germicide is effective against a wide range of pathogenic organisms and comprises 65-75 wt % of a monohydric alcohol selected from the group consisting of isopropyl, methyl, ethyl, n-propyl, n-butyl, tert-butyl, allyl, or mixtures thereof, and from about 4% to about 16% by weight of at least one polyhydric alcohol selected from the group consisting of propylene glycol, 1,3 propanediol, 1,2 butanediol, PEG 400, glycerol or 1,4 butanediol, or mixtures thereof in proportion by weight. The monohydric alcohol in the germicide provides the primary disinfecting or killing effect on the pathogenic organisms, while the polyhydric alcohol reduces the surface glaze formed by the monohydric alcohol and the surface tension formed by water or water-based body fluids, thereby enabling the disinfectant/antiseptic to kill pathogenic organisms and act equally effectively on a patient or inanimate surface without deleterious effects. The monohydric alcohol also lowers the flash point of the composition, soothes the skin, and slows the rate of evaporation. In this regard, the germicide is not a lubricant and does not include a warming constituent. The polyhydric alcohol, by reducing the rate of evaporation of the monohydric alcohol, essentially cools the surface by evaporative cooling.

U.S. Pat. No. 5,512,289 to Tseng et al. discloses a spermicidal anti-viral lubricant composition containing a water-soluble polymeric gel matrix comprising a hydroxyalkyl cellulose with 2 to 6 carbon atoms; an alkylphenoxypolyethoxyethanol spermicide: and a solubilizing moiety comprising a polyethoxylated non-ionic compound, such as polyethoxylated castor oil. The use of an alkylphenoxypolyethoxyethanol spermicide prevents collapse of the hydroxyalkyl cellulose gel. The addition of a solubilizer substantially prevents the collapse of the gel matrix and permits the gel matrix to maintain its properties in the composition. The spermicidal composition can be dispersed by use of a pharmaceutically acceptable vehicle, such as water, alcohols, e.g., ethanol, glycerin, propylene glycol, and mixtures thereof. A typical formulation of the spermicidal lubricant can include water 75.50 wt %, glycerin 17.00 wt %, hydroxyethyl cellulose 1.00 wt %, polyvinyl pyrrolidone 0.90 wt %, carboxymethyl cellulose 1.00 wt %, Nonoxynol-9 spermicide 2.00 wt %, polyethoxylated castor oil 2.00 wt %, methylparaben 0.20 wt %, sorbic acid 0.05 wt %, and citric acid 0.35 wt %. Since glycerin and glycol are already dissolved in water, the lubricant does not provide a warming effect.

U.S. Pat. No. 5,514,698 to Ahmad et al. discloses an antifungal vaginal cream composition. The composition has stable viscosity at human body temperature and comprises about 0.4 to 10.0% of an imidazole antifungal agent (of miconazole, econazole, terconazole, saperconazole, itraconazole, ketoconazole, and clotrimazole), about 1.0% to 5.0% of a fatty acid ester (isopropyl stearate, isopropyl myristate, isopropyl palmitate, and isopropyl laurate), about 1.0% to 25.0% of aliphatic alcohols (cetyl alcohol, stearyl alcohol and propylene glycol), about 2.0% to 5.0% of a surfactant (polysorbate 60 or polysorbate 80), about 0.02% to 0.20% of an antioxidant (butylated hydroxyanisole), a sufficient amount of inorganic base (NaOH or KOH) to adjust the pH range to a value of about 3.0 to 7.0, and water. This vaginal cream is not a lubricant and is expected to maintain its viscosity for prolonged time periods at body temperature. Even though propylene glycol is used in the composition, it is already mixed with water and, therefore, no warming effect takes place when the cream contacts body-generated moisture.

U.S. Pat. No. 5,649,825 to Ratkus discloses a dental root canal bacterialcidal lubricant, which allows the cleaning wires or files to move more freely when removing a nerve from a tooth. The composition reduces packing of tissue and dentin debris within the nerve cavity. This formulation is also resistive to decomposition during cold weather shipping, and includes cetyl alcohol, stearyl alcohol, sodium lauryl sulfate, stearic acid, propylene glycol, methyl paraben, propyl paraben, and butyl paraben in a purified water solution. The lubricant does not provide a warming effect.

U.S. Pat. No. 5,885,591 to Ahmad et al. discloses highly lubricious personal lubricant compositions containing one or more polyhydric alcohols, one or more water-soluble polymers derived from cellulose, water, and, optionally, preservatives and alkali metal or alkaline earth metal bases. These compositions can provide a vehicle for delivering medicaments for contraception and for the treatment and prevention of disease. The personal lubricant composition contains about 30% glycerin, about 5% propylene glycol, about 10% sorbitol, about 0.4% preservative, about 0.4% hydroxyethylcellulose, about 0.01% sodium hydroxide, and about 50% water. The composition has a lubricity of 33 to about 466. The personal lubricant composition attaches to mucous membranes and is not readily washed off. Since the composition already contains water, no heating or warming reaction occurs when the lubricant is applied to the body and the glycerin conies in contact with body-generated moisture.

U.S. Pat. No. 6,139,848 to Ahmad et al. discloses stable personal lubricant compositions containing at least one water-soluble polyhydric alcohol, a water-soluble polymer derived from cellulose, tocopherol or a tocopherol derivative, a nonionic water-soluble or dispersible emulsifier, and water. The lubricious, oily tocopherol or tocopherol derivative is present as an emulsion in the personal lubricant. The emulsifier preferably is polyalkylene emulsifier chosen from the group consisting of polyoxyethylene (20) sorbitan monostearate, commonly known as Tween 60 or Polysorbate 60, and polyethylene glycol ether of isocetyl alcohol (commonly known as Isoceteth 20 or Arlasolve 200, which is available from ICI Americas, Inc., New Castle, Del.). The water-soluble polyhydric alcohol serves to increase the lubriciousness of the compositions. The water-soluble cellulose-derived polymer serves to impart the desired slipperiness and viscosity to the composition. Water is desired in sufficient quantity to be delivered as moisture to the mucous membranes and to provide consistency and viscosity to the composition. The nonionic surfactants emulsify tocopherol, tocopherol acetate or other tocopherol derivatives into a microemulsion in which the internal or oil phase is reduced to very small globules measuring 2 or less than 2 microns in size. Due to their immensely small size, the emulsion globules do not coalesce and serve to assist in maintaining the homogeneity of the emulsions and to preserve physical stability of the compositions. Since the polyhydric alcohol (glycerin) is already combined with water, no heating or warming reaction occurs when the lubricant is applied to the body.

U.S. Patent Application Publication No. 2001/0039380 to Larson et al. discloses in vivo biocompatible acoustic coupling media comprising polyethylene oxide (PEO), at least one of polyalkylene glycols and polyhydric alcohols, and the balance water. Since water is already added to polypropylene alcohol, the exothermic reaction of mixing has already occurred, and no additional warming occurs when the lubricant is used.

U.S. Patent Application Publication No. 2003/0207772 and International Patent Application Publication No. WO 03/092651 to Ahmad et al. (see also U.S. Pat. App. Pub. No. 2003/0211161 and Intl Pat. App. Pub. No. WO 03/092652) disclose warming and nonirritating lubricating compositions containing polyhydric alcohols and an insulating agent. The polyhydric alcohol comprises glycerin, alkylene glycol, or a mixture thereof. The alkylene glycol is selected from the group consisting of propylene glycol, butylene glycol and hexylene glycol, whereas the polyethylene glycol is selected from the group consisting of polyethylene glycol 300 and polyethylene glycol 400. The insulating agent is selected from the group consisting of honey, isopropyl myristate and isopropyl palmitate. A warming action is created by the heat released during dissolution of the polyhydric alcohol in water, and the insulating agent retains the heat. The insulating agent is an essential part of the composition of the lubricant, and reduces the spreading capability of the warming lubricant.

U.S. Patent Application Publication No. 2003/0232090 and International Patent Application Publication No. WO 03/092650 to Ahmad et al. disclose warming and nonirritating lubricating compositions. Also disclosed are substantially anhydrous, warming, non-toxic and nonirritating lubricating compositions containing polyhydric alcohols, a gelling agent and, alternatively, a pH-adjusting agent for treating fungal and bacterial infections. The polyhydric alcohol comprises glycerin, alkylene glycol, polyethylene glycol, or a mixture thereof. The alkylene glycol is selected from the group consisting of propylene glycol, butylene glycol and hexylene glycol, whereas the polyethylene glycol is selected from the group consisting of polyethylene glycol 300 and polyethylene glycol 400. The insulating agent is selected from the group consisting of honey, isopropyl myristate and isopropyl palmitate. The use of insulating agents reduces the spreading capability of the warming lubricant.

U.S. Patent Application Publication No. 2004/0037911 to Letourneau et al. discloses a genital lubricating composition. The composition comprises (i) fatty acids and/or homeopathic dilutions of plant extracts and (ii) a physiologically acceptable carrier. According to a preferred embodiment, the genital lubricant composition comprises about 0.05% to about 0.5% hemp seed oil and a physiologically acceptable carrier so that it forms a lotion, a cream or a gel. The composition is particularly useful for use as a vaginal moisturizer, or as a personal lubricant for use prior or during sexual intercourse. The fatty acids can be hemp oil, linoleic (C18:2) acid or linolenic (C18:3) acid. The plant extracts include extracts from Caladium seguinum, Sepia officianalis, Lycopodium clavatum, and Onosmodium virginanium. The exact composition of the carrier is not disclosed. This lubricant does not have any polyhydric alcohol and does not produce a warming sensation.

None of these prior art patents or applications teaches the disclosed invention either singly or in combination. What is needed in the art of human body lotions is a non-aerosol combined lotion that can be applied without the known side effects of aerosol-applied liquids. Namely, aerosols spray the canister contents under too high a pressure for safe close skin contact. They also spray the contents at a much too cold temperature for sensitive skin areas. Nor is it taught to use the disclosed ingredients that include both neutricutical and skin conditioning properties in addition to the benefits of decreasing skin abrasion from frictional contact.

### SUMMARY OF THE INVENTION

The present invention has been developed in response to the present state of the art, and in particular, in response to the problems and needs in the art that have not yet been fully solved by currently available containers and lotions. Accordingly, the present invention has been developed to provide a formula for a personal topical body lotion and the method of combining components of the disclosed formula. Its is also not taught to dispense the lotion using a bag on valve (“BOV”) delivery system. The BOV delivery system allows the disclosed lubricant to be sprayed in any direction from any angle.

Some advantages of the presently disclosed invention involving the art of human body lotions is a non-aerosol combined lotion that can be applied without the known side effects of aerosol applied liquids. Namely, aerosols spray the canister contents under too high a pressure for safe close skin contact. They also spray the contents at a much too cold temperature for sensitive skin areas. Nor is it taught to use the disclosed ingredients that include both neutricutical and skin conditioning properties in addition to the benefits of decreasing skin abrasion from frictional contact.

There is disclosed a skin lotion application system, comprising a skin lotion including a lubricant, a bag, designed to contain the skin lotion, a container, designed to contain the bag and skin lotion, a pressurized gas, contained in the container and not in the bag, and a valve, coupled to the top of the bag to release skin lotion from the bag when the valve is actuated.

Another teaching of the invention is to use a skin lotion chemical composition, comprising:

a lubricant; b) a skin numbing ingredient like lidocane; c) a vitamin ingredient. wherein the vitamin is selected from the group consisting of vitamin A, C, E, D or B 12; d) an ingredient that causes human skin to tingle, wherein the tingling ingredient is selected from the group consisting of menthol and L-arginine ethyl ester; e) wherein the skin lotion includes about: 71% water, 0.070% sodium benzoate, 0.070% potassium Sorbate, 0.180% citric acidanhydrous (FCC, USP), 14.200% glycerine 99.7%, 14.200% propylene glycol USP, 0.140% Xanthan gun 80 mesh, and 0.140% guar gun 250 (food grade).

Another form of the invention may include a skin lubrication lotion that functions as a skin rubbing anti-friction barrier compound to prevent skin rashes from burning of skin from excessive rubbing, said lotion comprising: a) a lubricant; b) a skin numbing ingredient including lidocane; c) a vitamin ingredient, wherein the vitamin is selected from the group consisting of vitamin A, C, E, D or B12; d) a flavoring, selected from the group consisting essentially of cherry, orange, grape and peppermint; e) an ingredient that causes human skin to tingle, wherein the tingling ingredient is selected from the group consisting of menthol and L-arginine ethyl ester; and f) wherein the skin lotion further consisting essentially of about: 71% water, 0.070% sodium benzoate, 0.070% potassium Sorbate, 0.180% citric acidanhydrous (FCC, USP), 14.200% glycerine 99.7%, 14.200% propylene glycol USP, 0.140% Xanthan gun 80 mesh, and 0.140% guar gun 250 (food grade).

Reference throughout this specification to features, advantages, or similar language does not imply that all of the features advantages that may be realized with the present invention should be or are in any single embodiment or the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

### DETAILED DESCRIPTION OF THE INVENTION

For the purposes of promoting an understanding of the principles of the invention, reference will now be made to the exemplary embodiments. Wherein, each statement of an embodiment is to be considered independent or any other embodiment, despite any use of similar or identical language.

The disclosed lubricant is principally intended as a personal lotion or lubricant to supplement the body's natural lubrication fluids, to moisturize, and to enhance the comfort and ease of frictional skin contact activity. If the body does not produce a sufficient amount of natural lubrication, this can result in discomfort or irritation during contact activity. The disclosed lubricant is intended to provide increased lubricity so as to alleviate possible discomfort or irritation and allow persons with decreased production of natural lotion or lubricants to supplement said natural lubrication.

The disclosed liquid/lotion generally may be a non-sterile, water-based, white to off white, non-irritating, non-greasy, non-staining liquid or gel. Packaging of the disclosed liquid material may be a laminated aluminum bag, or any other suitable bag, with a personally controlled actuator and lid, referred to herein as the “bag on valve” or BOV delivery system. The liquid as disclosed herein has a viscosity such that it may be sprayed in an aerosol-like fashion using the BOV delivery system, thereby making use of the liquid more convenient and more appealing to users than other personal liquids that require application by use of hands or fingers, which some individuals may be adverse to.

There are currently some personal liquids commercially marketed which use non-aerosol-like spray containers. Liquids dispensing using such containers are not dispensed in a uniform manner. The disclosed lubricant is dispensed more evenly using the BOV delivery system than previously disclosed and marketed pump sprayable lubricants. The BOV delivery system also allows the liquid to be sprayed at any angle from any direction, which allows a user to more easily apply the disclosed lubricant to the desired area requiring application.

One embodiment of a BOV (bag on valve) delivery system uses an FDA-approved laminated aluminum bag, with a personally controlled actuator and lid, which allows the disclosed personal lubricant to be sprayed in an aerosol-like fashion. The bag of the BOV delivery system is placed into a canister or other similar container and pressure is added. The pressure or propellant can be in the form of air or a liquefied gas. After pressure is added the system is crimped shut. The disclosed personal lubricant product is then pressure-filled into the bag and sprays like an aerosol in a continuous fashion from any angle or direction, even when held upside down. The disclosed formula goes into the BOV system “neat” without any other chemicals mixed in. An example BOV system is pictured in the disclosed prior art of record, and any form or similar design known to those skilled in the art may be used.

In one embodiment, there is an empty canister, which will hold the pressurized gas. There is also the aluminum, or other material, elongated bag that will contain the liquid to be applied to the skin of a user. The bag is attached to the bottom of the spray valve, which is designed to eject the liquid contents from the bag and out of the container. There is also attached to the spray valve a top cover mechanism designed to seal the opening of the canister and retain the pressurized gas, and bag inside of the canister when the top is sealed shut to the canister in a known fashion.

In another embodiment of the disclosed personal lotion liquid lubricant, the compositions of the liquid are mainly water-based and include other components as described below. The methods of combining the described components for each of the preferred embodiments are also disclosed. The following examples serve to illustrate the composition and methods of the present invention. However, they are not presented in order to limit the scope of the invention in anyway.

### Example ONE

Table 1 below details the ingredients and their measurements used to create one embodiment of the personal lubricant formula disclosed herein. The ingredients are combined in the following manner to create the disclosed personal lotion/lubricant before adding the created liquid to the BOV system.

Add Glycerin and/or Propylene Glycol to a vessel to potentially be a lubricant base.

Add Guar Gum and Xanthan Gum slowly to pre-hydrate.

In a separate tank, add Water and Preservatives and mix for three minutes.

Add remaining ingredients to water tank, adding acid last.

Add water phase to vessel with Glycerin and Propylene Glycol.

Mix for 15 minutes on medium speed.

Let sit for 20 minutes before filling BOV bag to let air bubbles out.

TABLE 1

| | 0D-40 Flavored | | | |
|---|---|---|---|---|
| Supplier | Lubricant | 500 | Formula % | Kilo |
| 01 only | Water | 354.998 | 71.00% | 709.975 |
| Cargill | Sodium Benzoate | 0.355 | 0.07% | 0.70998 |
| Tate & | Citric Acid | 0.887 | 0.18% | 1.n494 |
| Lyle | | | | |
| Cargill | Potassium Sorbate | 0.355 | 0.07% | 0.70998 |
| Brenntag | Glycerine | 70.998 | 14.20% | 141.995 |
| Brenntag | Propelyne Glycol | 70.998 | 14.20% | 141.995 |
| TIC Gums | GuarGum | 0.710 | 0.14% | 1.41995 |
| TIC Gums | Xantham Gum | 0.710 | 0.14% | 1.41995 |
| | | 500 | 100% | 1000 |

### Example TWO

Table 2 below details the ingredients and their measurements used to create a second embodiment of the personal lotion/lubricant formula disclosed herein. The ingredients are combined in the following manner to create the disclosed personal lotion/lubricant before adding the liquid to the BOV system.

Table 2 makes reference to a Phase A and a Phase B. The Phase A ingredients include Water, Sodium Benzoate, Potassium Sorbate and Anhydrous, FCC, USP Citric Acid. The Phase B ingredients include Glycerine 99.7%, Propylene Glycol USP, Xanthan Gum 80 mesh, and food-grade Guar Gum 250.

TABLE 2

| | Item | Raw Material | % in | Batch Size |
|---|---|---|---|---|
| Phase | No. | Description | batch | 6600 |
| A | 10-0001 | Water | 71.000 | 4686.000 |
| | — | flavor | 0.000 | 0.000 |
| | — | sugar | 0.000 | 0.000 |
| A | 10-0090 | Sodium Benzoate | 0.070 | 4.620 |
| A | 10-0083 | Potassium Sorbate | 0.070 | 4.620 |
| A | 10-0014 | Citric Acid-Anhydrous, | 0.180 | 11.880 |
| | | FCC, USP | | |
| B | 10-0027 | Glycerine 99.7% | 14.200 | 937.200 |
| B | 10-0009 | Propylene Glycol USP | 14.200 | 937.200 |
| B | 10-0073 | Xanthan Gum 80 mesh | 0.140 | 9.240 |
| B | 10-0688 | Guar Gum 250 (FOOD | 0.140 | 9.240 |
| | | GRADE) | | |
| | | | 29.000 | 6600.000 |
| | | qs-Water: | 71.000 | |
| | | Formulation Total: | 100.000 | |

The Phase A ingredients are first added to a mixing vessel. While Phase A is processing, Phase B is premixed together. When Phase B is completely wetted and uniform it is added to Phase A. The combined phases are mixed until thickened and have uniform consistency.

Another embodiment of the invention may include the solution to have a neutral pH, and more specifically, a pH of about 6.5 to 7.5 preferably.

Yet a further feature of the invention may be to include additional ingredients of neutricuticals and/or vitamins. Specifically, it is god to include vitamins that are good for the skin, such as vitamin A, C, E, D, B 12 and others that are absorbable through or into the skin. Another useful ingredient could be to include adding aloe into the liquid. One skilled in the art would find it beneficial to include any number of other beneficial ingredients, such as those found in anti-oxidant related ingredients. Additionally, naturally occurring or artificial flavoring is added to the solution lubricant liquid, to enhance the flavor of the solution, like cherry, or orange flavors.

It is also contemplated in another embodiment to include ingredients to cause nerve numbing to the area. Specifically, lidocane is a known ingredient that causes numbing of the skin upon topical application to the skin. Other ingredients would be known by skilled artisans, and are included in this disclosure to include for the purpose of causing a deadening of the nerves, or other such effects to cause numbing sensations.

Another contemplated class of ingredients are those that cause tingling of the skin after application. Such ingredients known to cause tingling could include menthol, L-arginine ethyl ester, or other known compounds or liquids that are sprayable and cause tingling to the skin.

Thus, while the present invention has been fully described above with particularity and detail in connection with what is presently deemed to be the most practical and preferred embodiment of the invention, it will be apparent to those of ordinary skill in the art that numerous modifications, including, but not limited to, variation in size, materials, shape, form, function and manner of operation, assembly and use may be made, without departing from the principles and concepts of the invention as set forth in the claims.

What is claimed is:

1. A skin lotion chemical composition, comprising:
a) a lubricant;
b) a skin numbing ingredient including lidocane;
c) a vitamin ingredient, wherein the vitamin is selected from the group consisting of vitamin A, C, E, D or B 12;
d) an ingredient that causes human skin to tingle, wherein the tingling ingredient is selected from the group consisting of menthol and L-arginine ethyl ester; and
e) wherein the skin lotion includes about: 71% water, 0.070% sodium benzoate, 0.070% potassium Sorbate, 0.180% citric acidanhydrous (FCC, USP), 14.200% glycerine 99.7%, 14.200% propylene glycol USP, 0.140% Xanthan gun 80 mesh, and 0.140% guar gun 250 (food grade).

2. A skin lotion chemical composition, consisting essentially of:
a) a lubricant;
b) a skin numbing ingredient including lidocane;
c) a vitamin ingredient, wherein the vitamin is selected from the group consisting of vitamin A, C, E, D or B 12;
d) an ingredient that causes human skin to tingle, wherein the tingling ingredient is selected from the group consisting of menthol and L-arginine ethyl ester; and
e) wherein the skin lotion further consisting essentially of about: 71% water, 0.070% sodium benzoate, 0.070% potassium Sorbate, 0.180% citric acid-anhydrous (FCC, USP), 14.200% glycerine 99.7%, 14.200% propylene glycol USP, 0.140% Xanthan gun 80 mesh, and 0.140% guar gun 250 (food grade).

3. A skin lubrication lotion that functions as a skin rubbing anti-friction barrier compound to prevent skin rashes from burning of skin from excessive rubbing, said lotion comprising:
a) a lubricant;
b) a skin numbing ingredient including lidocane;
c) a vitamin ingredient, wherein the vitamin is selected from the group consisting of vitamin A, C, E, D or B 12;
d) a flavoring, selected from the group consisting essentially of cherry, orange, grape and peppermint;
e) an ingredient that causes human skin to tingle, wherein the tingling ingredient is selected from the group consisting of menthol and L-arginine ethyl ester; and
f) wherein the skin lotion further consisting essentially of about: 71% water, 0.070% sodium benzoate, 0.070% potassium Sorbate, 0.180% citric acid-anhydrous (FCC, USP), 14.200% glycerine 99.7%, 14.200% propylene glycol USP, 0.140% Xanthan gun 80 mesh, and 0.140% guar gun 250 (food grade).

4. The invention of claim 1, further including a flavoring, selected from the group consisting essentially of cherry, orange, grape and peppermint.

5. The invention of claim 2, further including a flavoring, selected from the group consisting essentially of cherry, orange, grape and peppermint.

6. The invention of claim 1, further including placing the skin lotion chemical composition into a bag on valve container

7. The invention of claim 2, further including placing the skin lotion chemical composition into a bag on valve container.

8. The invention of claim 3, further including placing the s skin lubrication lotion into a bag on valve container.
